(19) United States
(12) Patent Application Publication
(10) Pub. No.: US 2025/0120671 A1
(43) Pub. Date: Apr. 17, 2025

(54) SYSTEMS AND METHODS FOR PROVIDING AUGMENTED ULTRASONOGRAPHY WITH AN IMPLANTED ELECTRONIC DEVICE

(71) Applicant: The Trustees of Columbia University in the City of New York, New York, NY (US)

(72) Inventors: Kenneth L. SHEPARD, Ossining, NY (US); Prashant MUTHURAMAN, New York, NY (US); Yihan ZHANG, New York, NY (US)

(21) Appl. No.: 18/990,861

(22) Filed: Dec. 20, 2024

Related U.S. Application Data
(63) Continuation of application No. PCT/US2023/025861, filed on Jun. 21, 2023.
(60) Provisional application No. 63/354,076, filed on Jun. 21, 2022.

(51) Int. Cl. A61B 8/08

(52) U.S. Cl. CPC A61B 8/0833

(57) ABSTRACT

Exemplary apparatus and method can be provided for use with ultrasound imaging. The apparatus can include at least one implantable device which is configured to communicate with an ultrasound imaging system, be located by the ultrasound imaging system using ultrasound signals generated thereby to generate a location of the implantable device(s), and transmit data to the ultrasound imaging system from the location of the implantable device(s) that was located by the ultrasound imaging system. Further, it is possible to implant, into a structure, at least one device which is responsive to ultrasound signals generated by the ultrasound imaging system, locate the device(s) within the structure by the ultrasound imaging system using the ultrasound signals to generate a location of the device(s) within the structure, and transmit data to the ultrasound imaging system from the location of the device(s) that was located by the ultrasound imaging system.

### CROSS REFERENCE TO RELATED APPLICATION(S)

This application relates to and claims the benefit of priority from U.S. Provisional Patent Application No. 63/354,076, filed Jun. 21, 2022, the entire disclosure of which is incorporated herein by reference in its entirety.

### STATEMENT REGARDING FEDERALLY SPONSORED RESEARCH

This invention was made with government support under HR0011-15-2-0054 and D20AC00004 awarded by the Defense Advanced Research Projects Agency. The government has certain rights in the invention.

### FIELD OF THE DISCLOSURE

The present disclosure relates to ultrasonography, and more particularly to systems and methods for providing augmented ultrasonography with implanted electronic device(s), including Complementary Metal Oxide Semiconductor (CMOS) mote(s).

### BACKGROUND INFORMATION

There have been decades of technological advances in the field of ultrasound medical diagnostic imaging. With pioneering work¹ dated back to the 1950s, medical ultrasound, or ultrasonography, has evolved to become a routine imaging procedure for real-time tomography. Compared to other medical tomographic techniques, it has many advantages because of the unique properties of ultrasound waves. In addition to being non-ionizing, the low acoustic energy loss in tissue (approximately 0.5˜1 dB/cm/MHz^{2,3}) allows for significant imaging depth while high spatial resolution is possible because of the short wavelength characteristic of acoustic energy in tissue (about 0.5 mm at a typical carrier frequency of 3 MHz). Imaging frame rates are typically in excess of 25 frames per second (fps) due to advances in imaging hardware and software.

At the same time and motivated by many of the same properties that make it attractive for diagnostic imaging, ultrasound is emerging as a means for powering and communicating with implanted medical devices⁴⁻⁸. Wireless powering is attractive because it eliminates the need for batteries, which introduce extra safety concerns and consume considerable volume. What is particularly advantageous for ultrasound in these applications is that attenuation in tissue is significantly less for ultrasound when compared with electromagnetic waves at comparable wavelengths⁹. Wavelength is important because it determines minimum antenna sizes and, consequently, the size of the implanted devices themselves. The low absorption of ultrasound in tissue also supports average power densities as high as 7.2 mW/mm² without deleterious effects¹⁰. For electromagnetic radiation, the specific absorption ratio (SAR) limits incident average power densities to less than 0.22 mW at 1.6 GHz, for example, in the case where an antenna is positioned at the tissue surface¹¹.

To achieve μW-level power budget, previous devices based on ultrasound for wireless powering rely on focused, almost continuous ultrasound “beams”. Traditional digital modulation techniques can be then implemented on top of such beams for data transmission, like on-off keying (OOK)¹², and pulse-width modulated amplitude-shift keying (PWM-ASK)^{6,13}. Uplink data can be captured in the acoustic domain once the device is properly powered, in the form of either backscattered incident ultrasound^{5,14,15} or active transmission of ultrasound energy from the implant¹². In all of these implementations, accurate knowledge of the implant location is required for power delivery and communication. This location information can be obtained either through an additional imaging session prior to the operation of the implant or a designed localization process on top of normal operation. One such designed process uses a sharp ultrasound pulse emitted from the implant to back calculate the delays in each transmitter element for focusing, but the implant needs to harvest some power before the chirp can be emitted¹⁶⁻¹⁸. Another example extracts this delay information from higher order harmonics of the implant's reflected acoustic waves¹⁹; but without an active signature, harmonics in the tissue, long used in ultrasound harmonic imaging²⁰, can potentially shadow this information. Even if the implant can be localized, in many in vivo settings, continuous movement due to muscle activity, heartbeat, and respiration can perturb device location, requiring frequent recharacterization to maintain focus.

Thus, it may be beneficial to provide exemplary cavity-separated multi-nanopore (CSMP) device and method which can facilitate protein sequencing, which can overcome at least some of the deficiencies described herein above.

### SUMMARY OF EXEMPLARY EMBODIMENTS

The following is intended to be a brief summary of the exemplary embodiments of the present disclosure, and is not intended to limit the scope of the exemplary embodiments.

According to various exemplary embodiments of the present disclosure, the operation of the exemplary specifically designed ultrasound mote²¹ can be integrated and/or utilized with medical sonography, facilitating such combination to be biogeographically located in the image, to be powered by the ultrasound imager, and to communicate back through the image. For example, the mote (e.g., the implantable device) can stay continuously powered when located within the ultrasound imaging sensor's field of view (FoV). Bi-directional data transmission is established synchronously with the imaging frame rate and the uplink information (from implant to the imager) can be retrieved during image reconstruction. Multiple motes can be deployed within a given FoV with non-interfering parallel operation, as digital uplink data signatures are spatially separated in the reconstructed image. Such exemplary digital data signatures give the motes contrast relative to the surrounding, continuously moving biological environment and can be used to localize them at the resolution of the resulting B-mode image. These exemplary sensors facilitate an “augmented ultrasonography”, delivering real-time, biogeographically related physiological information from multiple locations on top of a traditional ultrasound tomographic imaging.

The use of imaging-mode ultrasound can impose a restricted power budget on the motes, typically nano-Watts (nW) or below, as well as a data rate limited to the imaging frame rate, which is typically in the range of 10s of Hz. Fortunately, these data rates are sufficient for most physiological parameters, such as temperature, blood pressure, pH, and most biomarkers. There are many CMOS sensor systems available that operate with nW or sub-nW power consumption that would be compatible with this power envelope at the bandwidths of interest^{14,22-24}. Even in the absence of integrated sensing, motes such as these transmitting unique identifiers can find application in real-time tracking of surgical sites, such as in intramedullary nailing^{25,26}.

Indeed, modern clinical practice benefits significantly from imaging technologies and much effort is directed toward making this imaging more informative through the addition of contrast agents or reporters. Here, an exemplary design of a battery-less integrated circuit mote acting as an electronic reporter during medical ultrasound imaging can be provided. When implanted within the field-of-view of a brightness-mode (B-mode) ultrasound imager, this exemplary mote can transmit information from its location through backscattered acoustic energy which is captured within the ultrasound image itself. It is possible to characterize the operation of such motes in vitro and in vivo. Performing with a static power consumption of less than 57 pW, the motes operate at duty cycles for receiving acoustic energy as low as 50 ppm. Motes within the same field-of-view during imaging have demonstrated signal-to-noise ratios of more than 19.1 dB at depths of up to 40 mm in lossy phantom. Physiological information acquired through such motes, which is beyond what is measurable with endogenous ultrasound backscatter and which is biogeographically located within an image, has the potential to provide an augmented ultrasonography.

To that end, an exemplary system according to the exemplary embodiments of the present disclosure can be provided for use with ultrasound imaging. The exemplary apparatus can include at least one implantable device which is configured to (i) communicate with an ultrasound imaging system, (ii) be located by the ultrasound imaging system using ultrasound signals generated thereby to generate a location of the implantable device(s), and (iii) transmit data to the ultrasound imaging system from the location of the implantable device(s) that was located by the ultrasound imaging system.

Further, an exemplary method can be provided for performing ultrasound imaging according to the exemplary embodiments of the present disclosure. With the exemplary method, it is possible to implant, into a structure, at least one device which is responsive to ultrasound signals generated by the ultrasound imaging system, locate the device(s) within the structure by the ultrasound imaging system using the ultrasound signals to generate a location of the device(s) within the structure, and transmit data to the ultrasound imaging system from the location of the device(s) that was located by the ultrasound imaging system.

In further exemplary embodiment of the present invention, it is possible to power the implantable device(s) by the ultrasound imaging system. The implantable device(s) can be continuously powered by the ultrasound imaging system when the implantable device(s) is located within a field of view of the ultrasound imaging system. In addition, it is possible to transmit the data to the ultrasound imaging system by the implantable device(s) using modulation. The implantable device(s) can be further configured to be implanted into the structure or a body through an injection. The implantable device(s) can be sized and configured to be injected into the structure of the body using a syringe.

According to still further exemplary embodiments of the present disclosure, it is possible to receive ultrasound signals from the ultrasound imaging system by the implantable device(s) so that the ultrasound imaging system ascertains the location of the implantable device(s). The transmission of the data to the ultrasound imaging system and the receipt of the ultrasound signals can be synchronous. The receipt of the ultrasound signals by the implantable device(s) from the ultrasound imaging system can be performed using a further modulation. The further modulation of the received ultrasound signals can be performed using at least one amplitude shift key, and the modulation of the transmission of the data can be performed using at least one load shift key back.

In a further exemplary embodiment of the present disclosure, the implantable device(s) can be a CMOS device or a monolithically-integrated device (e.g., a monolithically-integrated piezoelectric transducer). There can also be a plurality of implantable devices. It is possible to deploy each of the plurality of the implantable devices within a field of view of the ultrasound imaging system and providing a non-interfering parallel operation. Additionally or alternatively, each of the plurality of the implantable devices can be implanted at different locations within the structure.

According to yet an additional exemplary embodiment of the present disclosure, the transmission of the data from the e implantable device to the ultrasound imaging system can be performed using a backscattered acoustic energy.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an exemplary illustration of how ultrasonography-compatible motes interact with ultrasound imaging;

FIG. 2a is an illustration of an operating principle of B-mode sonography that guides the physical layer implementation;

FIG. 2b is a diagram of an exemplary simulated ultrasound beam profile with beam forming from 31 transducers focused to a depth of 15 mm, showing the peak pressure detected at each spatial location in the beam;

FIG. 2c is a diagram of a verified in the measured hydrophone data at different depths;

FIG. 3a is a block diagram of the imaging system and the designed mote according to an exemplary embodiment of the present disclosure;

FIG. 3b is a diagram of the timing of the signals used to implement the physical layer according to an exemplary embodiment of the present disclosure;

FIG. 3c is a diagram of an exemplary rectifier utilized with the exemplary embodiments of the present disclosure;

FIG. 3d is a diagram of an exemplary voltage regulator for circuit blocks utilized with the exemplary embodiments of the present disclosure;

FIG. 3e is a set of diagrams for circuits for clock recovery;

FIG. 3f is a diagram for an exemplary data recovery circuit and providing for the number of cycles in the first pulse that meet the threshold for clock recovery within each pulse packet;

FIG. 3g is a diagram for an exemplary the uplink backscatter modulator which can be used to maximize the modulation depth by shorting the piezoelectric element through MBS when transmitting an uplink bit 0;

FIG. 4a is an illustration of a fabricated integrated circuit with a die micrograph in which a mote ASIC is fabricated using a TSMC 180 nm MSRFG 1P6M process according to an exemplary embodiment of the present disclosure;

FIG. 4b is an illustration of an exemplary intact processed fuse indicating a bit;

FIG. 4c is an illustration of an exemplary fully integrated implantable mote which includes 1 piezoelectric crystal on a 2 mm×2 mm×0.66 mm printed circuit board (“PCB”);

FIG. 5a is an illustration of an exemplary setup in which an exemplary delay-and-sum procedure is used in image reconstruction for uplink data identification;

FIGS. 5b and 5c are a set of illustrations of how a ratio between the signal power and the noise power, or signal-to-noise ratio (SNR) varies as the mote moves away from the source transducer;

FIG. 6a is an illustration of an exemplary configuration which includes motes within an imaging application, chicken breasts are hand sliced into about 1-cm-thick layers and soaked in castor oil to act as a tissue phantom, with two motes between the chicken-breast layers with approximate mote locations overlaid;

FIG. 6b is a B-mode image captured by the linear array transducer in which a temporal average removal is used to reveal locations with abrupt, frame-to-frame intensity changes (plotted verses frames at the bottom);

FIG. 6c is an illustration of an exemplary timing diagram providing how the transducer array first sends out a “QUERY ID” instruction, to which all the motes respond by sending their specific identifiers (IDs) back;

FIG. 6d is an illustration of the devices implanted into the lower hind limb of a mouse, while the same B-mode imaging system and imaging configuration used for the phantom example is used to communicate with the mote;

FIG. 6e is an exemplary B-mode movie and data pattern from the mote for this in-vivo experiment; and

FIG. 6f is an exemplary detailed timing diagram showing the interaction between the imaging system and the implanted mote which is provided to the diagram of FIG. 3c.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended paragraphs.

### DETAILED DESCRIPTION OF EXEMPLARY EMBODIMENTS

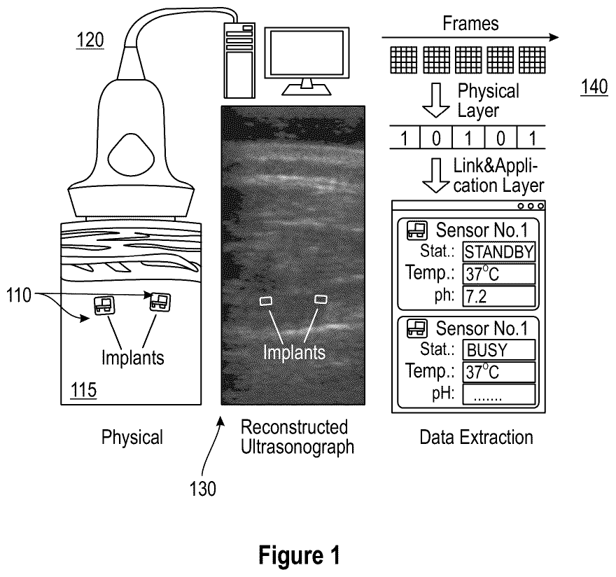

FIG. 1 shows how ultrasonography-compatible motes interact with ultrasound imaging. It is possible to consider brightness-mode (B-mode) imaging in this example, although it is noted that these exemplary devices can operate with any pulse-echo-mode ultrasound imaging technique. This exemplary illustration is provided, e.g., after the motes 115 can be physically implanted 115, they can be identified in an ultrasonography using an ultrasonography system 120, and data can be transmitted bidirectionally between the imager and the mote to retrieve real-time physiological information. When placed deep in tissue and within the FoV of an ultrasound imaging probe, the implant appears due to a local mismatch in acoustic impedance as a bright spot in the reconstructed image 130. Unlike a passive physiological structure, however, the mote 110 modulates its backscatter from frame to frame 140, making the motes easily identifiable in the ultrasound image. This modulation also provides the data uplink. The data downlink is, in turn, implemented by changing the width of the ultrasound pulses emitted from the probe during imaging. Instructions and measured physiological data can be sent through this bi-direction data link, with predefined data transmission protocols. Thus, from a communications perspective, these ultrasonography-compatible motes 110 can establish a physical layer information channel, upon which data can be delivered reliably for sensing applications at higher protocol layers.

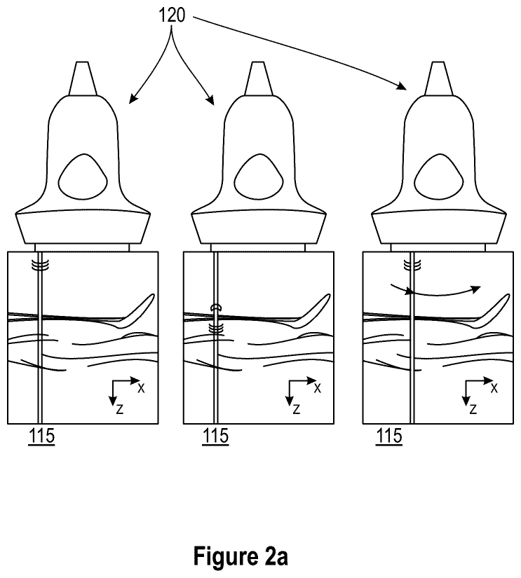

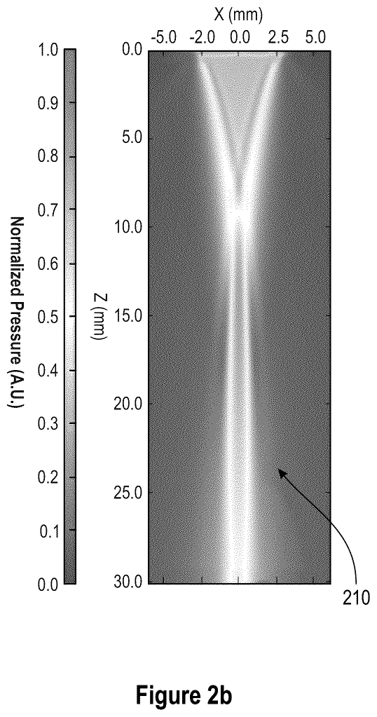

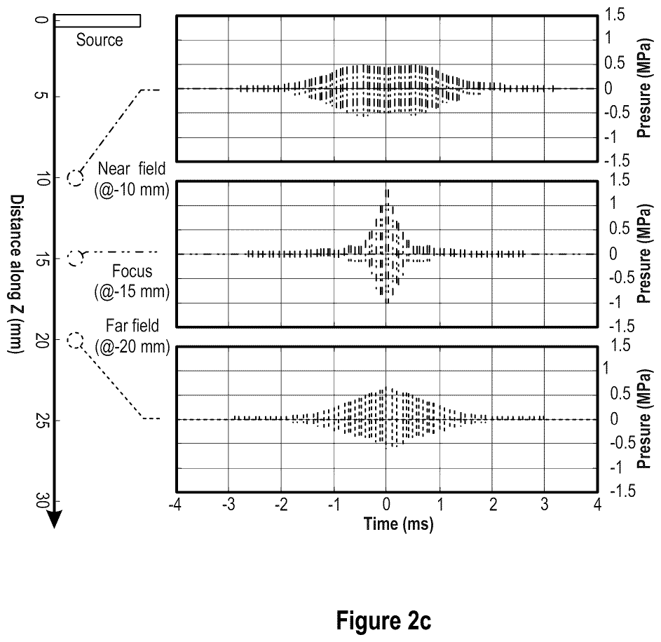

Exemplary B-Mode Sonography and Challenges in the Physical Layer. FIG. 2a shows an exemplary illustration of the pulse-echo imaging operating principle of B-mode sonography that guides the physical layer implementation. Traditional B-mode image frames are generated by scanning a focused ultrasound pulse over the field-of-view and measuring the back-reflected echoes²⁷. The depths of all the impedance discontinuities along the path of traversal of the pulse are determined by measuring the delay between the received echoes and the source pulse. Each pulse travels along a line in the axial (or z) direction at a certain transverse (or x) coordinate, as shown in FIG. 2a, known as a scan line or beam. In a linear array probe, this beam forming can be implemented through phased pulsing of multiple elements around the transverse position of interest. Scanning in the transverse direction is implemented by changing this phasing or by changing the elements employed to produce a given beam. In the exemplary case according to the exemplary embodiment of the present disclosure, it is possible to employ, e.g., a Verasonics L12-3V linear array probe, a 192-element linear ultrasound transducer array, in which, e.g., 31 elements at a time can be phased to produce a focused, z-directed pulse at a given position in x along the transducer array, which is then scanned to produce a frame. For example, to maintain a frame rate of 50 Hz, for example, a total of 192 pulses can be emitted at a 100-μs time interval for each frame, and the selection of the 31 transducers is shifted by one in the x direction for each pulse.

Harvesting energy in the context of B-mode sonography can be performed from an ultrasound energy that can be very sparse in time. For the frame rate of 50 fps employed in the exemplary case according to the exemplary embodiment of the present disclosure, each 1-μs-long pulse provides power to the mote with a duty cycle of 50 parts per million (ppm). The energy that can be available for power harvesting scales as approximately 0.33 pJ/(kPa)² for each pulse with energy at a MHz-level center frequency. The energy harvesting circuits should be able to respond to these frequencies without significant static power consumption from biasing and leakage currents. Energy storage in the form of decoupling capacitors can be required to maintain continuous powering. At a 50-ppm duty cycle and a minimum incident pressure of 400 kPa, which is typical for the imaging studies here, e.g., the decoupling capacitance should be at least 100 pF. Making the decoupling capacitance larger than minimum value, however, extends the start-up time, the time interval between when the imaging probe first find the mote to the time the mote generates a distinguishable data signature, since a supply voltage can reach at least 1.2 V before the mote can begin to operate.

An exemplary data transmission from the mote to the imager can take advantage of the way B-mode imaging is performed. For example, FIG. 2b shows an exemplary embodiment in which the simulated ultrasound beam profile 210 with beam forming from 31 transducers (e.g., linear array transducers) is focused to a depth of 15 mm, showing the peak pressure detected at each spatial location in the beam. In the near-field region as determined by the transducer and focal depth, the ultrasound energy is more diffuse. Beyond the focal depth in the far-field, the energy distribution pattern becomes narrower but still depends on the distance from the probe. This finite spatial extent of the ultrasound beam can mean that each mote can both receive and back-scatter many pulses in each frame, which can be denoted as a “pulse packet.”

This can be verified in the measured hydrophone data which provides a measured pulse envelope at different distances from the source linear array that is shown in the exemplary graphs of FIG. 2c at different depths, in which the same Verasonics linear array probe is used with beam forming configurations substantially identical to the simulation conditions as provided in the illustration of FIG. 2b. The exemplary amplitude distribution of the pulses in a pulse packet is a strong function of the relative distance between the imaging probe and the implant of interest. This imperfect beam forming is universal in all practical ultrasound imaging systems. As a result, motes within the FoV will find the ultrasound waveform different for the same downlink bit and backscatter a different number of pulses for the same uplink bit depending on its depth.

To ensure that the mote can establish a reliable data link when positioned anywhere within the FoV, the downlink data as transmitted by the imaging transducer can be maintained constant for the entire frame. At the mote side, backscattered data can also be maintained constant for each imaging frame. This can reduce or eliminate any dependency data transmission may have on the exact shape of the pulse packet, since every pulse within each packet carries the same downlink or uplink bit. The data rate in this case is determined by the frame rate.

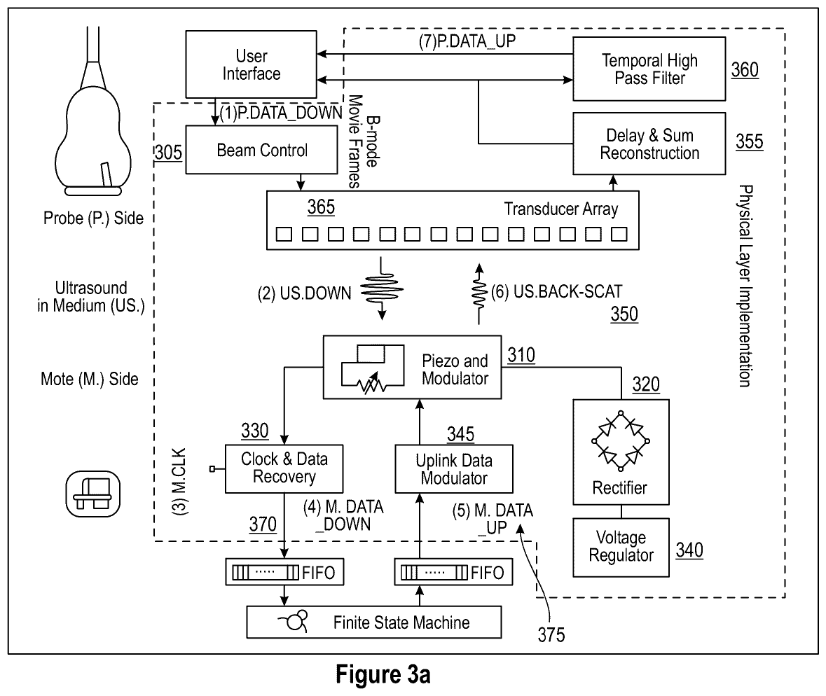

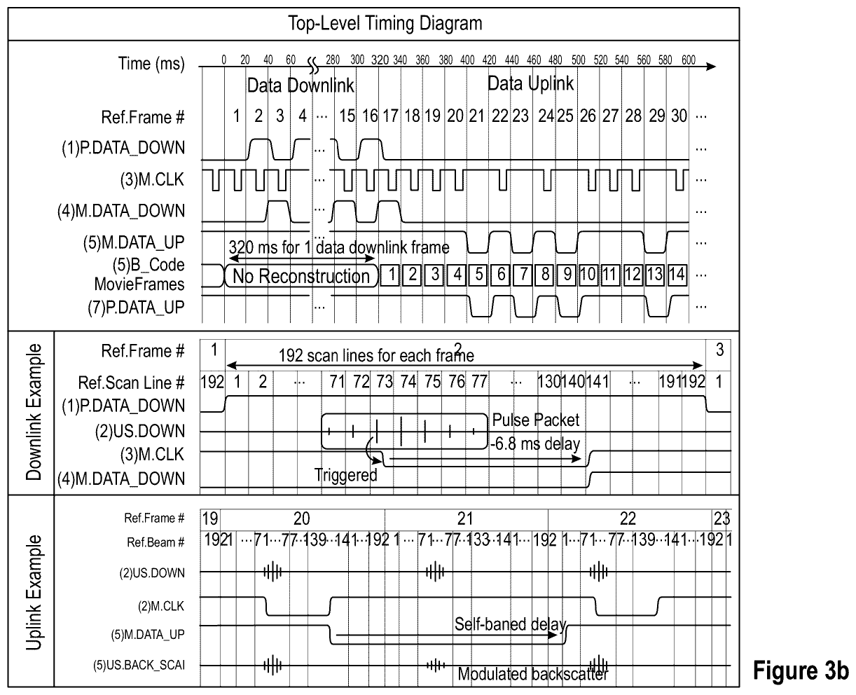

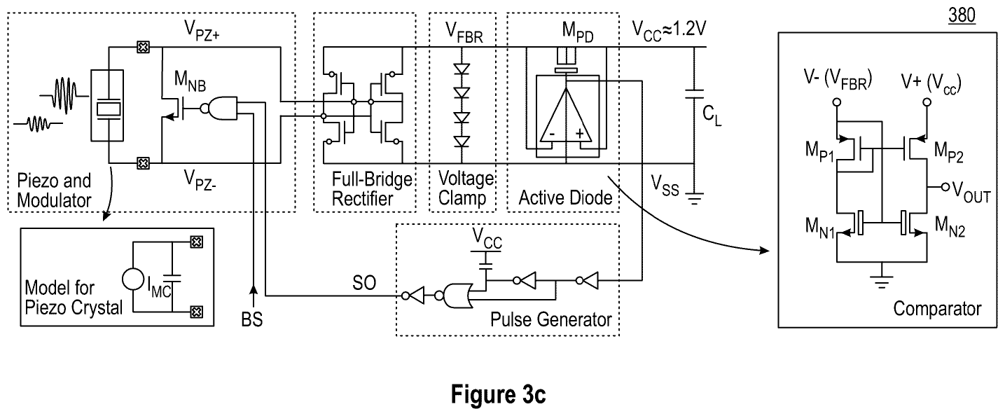

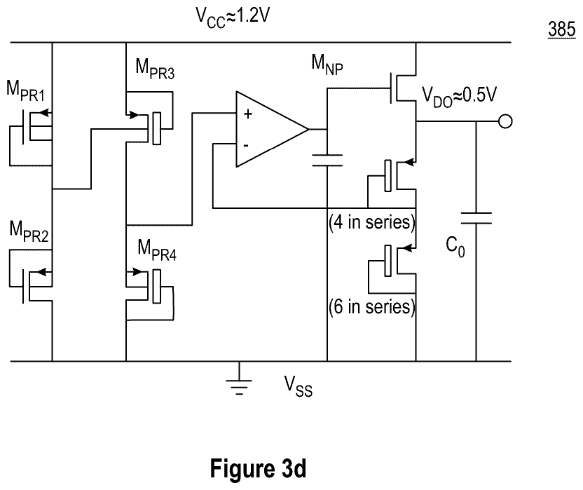

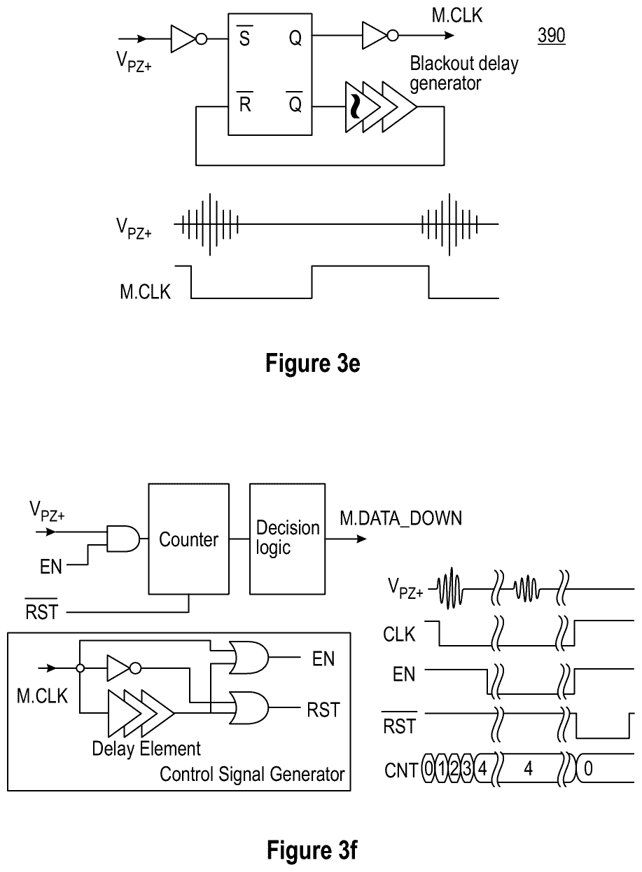

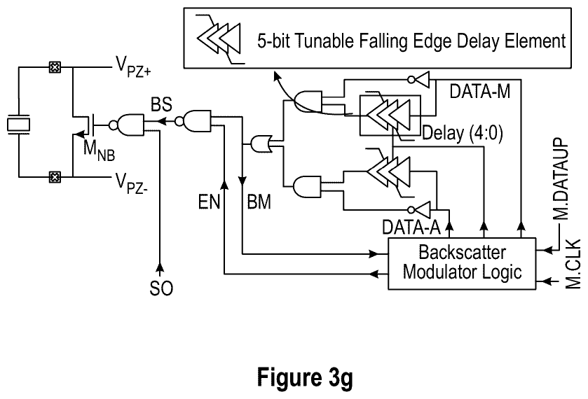

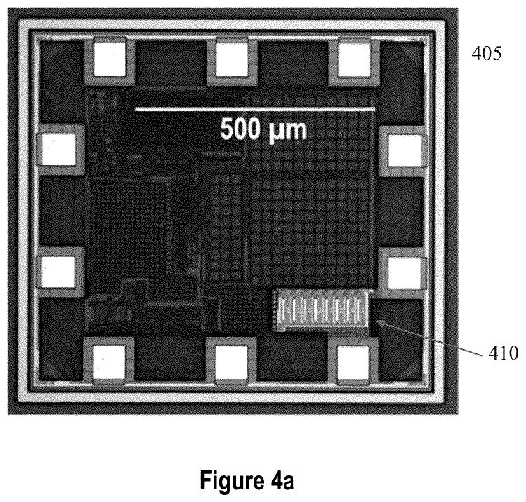

Exemplary System Level Design. FIG. 3a shows an exemplary block diagram of the imaging system and the designed mote according to an exemplary embodiments of the present disclosure. The timing of the signals used in the physical layer implementation is illustrated in FIG. 3b. For example, using standard imaging hardware, beam control 305 can be customized using software to facilitate different pulse widths to carry the downlink data. On the mote, the rectifier 320 can harvest energy from the imaging ultrasound, and the voltage regulator 340 needs to use the rectified power to provide proper supply voltage regulation for the rest of the mote. Each mote in the field-of-view can observe pulse packet at different times in the frame period depending on its lateral coordinate and receives different pulse packet shapes depending on its depth from the transducer array 365. In FIG. 3b, e.g., a pulse packet assumed between the 71st and 77th scanline out of a total of 192 is illustrated as an example, which corresponds to a mote found somewhere between the 71st and 77th scanline in the lateral coordinate.

Such exemplary received pulses can be used to recover a clock 330 of FIG. 3a synchronized to the frame rate, which can be used for chip operation. This clock facilitates the mote to synchronously recover the downlink data 370 and backscatter the uplink data, e.g., both being 16-bit data packets. The backscattered ultrasound 350 can be picked up by the imaging transducer array as a part of the B-mode echo, which, after standard delay-and-sum image reconstruction, forms the B-mode movie. The modulated uplink data 375 provided from an uplink data modulator 345 can thus translate to a frame-to-frame intensity change at the location of the mote. A software-defined temporal high-pass filter 360 can be used on the reconstructed B-mode movie provided from the delay and sum reconstruction procedure/module 355 to assist data visibility. A link-layer frame and an application-layer protocol, described in more detail below, complete the communication structure.

Turning to FIG. 3b, this figure shows the top level timing diagram of key signals according to the exemplary embodiments of the present disclosure. As shown in FIG. 3b, the frame rate is assumed to be 50 Hz, thereby leading to an M. CLK(3) period of 20 ms. The M.DATA_DOWN(4) is one cycle delayed compared to P.DATA_DOWN(1) due to an on chip gated flip flop. The downlink data packets consist of 16 bits each which lead to a reconstruction blackout time of 320 ms for 1 data downlink frame. Following a processing delay which is assumed as 4 cycles here, the M.DATA_UP(5) is asserted by the Finite State Machine Core which leads to the P.DATA_UP(7) signal received at the probe end.

The Downlink example of FIG. 3b illustrates the timing of relevant signals involved in downlink data capture in a single frame. Each frame consists of 192 scan lines which correspond to different lateral coordinates. The mote is assumed to be between the 71st and 77th scanline. When a pulse packet is sent by the probe, it triggers a falling edge in the M.CLK(3) which automatically returns to level 1 after a 6.8 ms delay. The M.DATA_DOWN (4) is asserted after this rising edge of the clock. The Uplink example shows the timing of relevant signals involved in an Uplink data transfer. The M.DATA_UP (5) is de-asserted at a rising edge of the M.CLK(3) and returns to level 1 after a self-tuned delay. The US_DOWN(2) that arrives during this down time is modulated and backscattered as US.BACK_SCAT(6).

Exemplary Rectifier Design and Power Regulation. To harvest heavily duty-cycled ultrasound energy, it is possible use an active rectifier (show in detail in FIG. 3c) based on a published design²⁸ because of its superior on-off ratio. However, in this exemplary design, static power from biasing can significantly reduce the effective efficiency when operated at duty cycles at the level of ten ppm. In order to reduce the power consumption required to bias the continuous-time comparator operating at MHz-level switching speeds, it is possible to modify such previous design sown in FIG. 3c with a dynamic biasing scheme 380 according to an exemplary embodiment of the present disclosure, in which the gates of the current source pair M_{N1} and M_{N2} are connected to the inverting input of the comparator, which is connected, in turn, to the output of the full-bridge rectifier (V_{FBR}). In this exemplary way, the comparator can deliver a sub-μs decision time, with negligible static power consumption when no ultrasound energy is present.

Many previous efforts have relied on operating the piezoelectric transducer at the resonance of the piezoelectric crystal for maximum power delivery^{28,29}. However, those approaches introduce two problems. First, a resonant power delivery requires a high-quality mechanical resonance, which, in turn, demands highly mismatched boundary conditions around the piezo transducer of interest. This can be achieved by adding an air-pocket-based backing structure underneath the piezo transducer¹², at the cost of a more complicated packaging as well as an increased mote volume. Second, significant impedance changes typically occur within the approximately 100-kHz bandwidth around the package-defined resonance frequency. As a result, the amount of power delivered strongly depends on the frequency tuning resolution of the imaging transducer array, making the motes difficult to power in practice.

For these reasons, according to exemplary embodiments of the present disclosure, it is possible to assume a capacitive input impedance, a typical case when the piezo transducer is surrounded by non-optimized mechanical boundary conditions. Resonance is still possible in the electrical domain with the help of an inductor for conjugate matching, although it is possible to choose not to do this because it would significantly increase implant volume. Instead, to boost the power harvesting without resonance, it is possible to employ a “switch-only” rectifier, a technique commonly used in low-frequency mechanical energy harvesting circuits³⁰ but adapted here for carrier frequencies in the MHz regime for reliable, nW-level power harvesting. Such an approach consumes little on-chip area while relaxing the requirement for a high-quality mechanical resonance from the piezoelectric element. These exemplary benefits together can facilitate an efficient off-resonance power harvesting and the potential to scale to sub-0.1-mm³ range implant volumes with piezoelectric transducer integration^{14,31}.

The exemplary rectifier according to exemplary embodiments of the present disclosure can generate, e.g., an 1.2-V dc supply, V_{CC}, which can be clamped by the voltage limiter, and can be used to power circuits which should have enough performance to track the MHz-level ultrasound carrier. A 0.5-V V_{DD} is generated from V_{CC} with a linear voltage regulator 385 (shown in FIG. 3d) for circuit blocks, such as delay generators and digital circuits, that may require an accurate supply voltage, but operate at lower performance requirements, typically synchronized to the 50-Hz frame rate, for dynamic power reduction. A pW-level trim-free voltage reference circuit³² is used to produce a supply-independent and temperature-independent 0.5-V reference voltage from V_{CC} for this linear regulator.

Exemplary Clock Synchronization and Bi-Directional Data Telemetry. Once sufficient energy is harvested for start-up, the mote establishes synchronized bi-directional data transmission with the imager. FIG. 3e shows exemplary circuit(s) 390 for clock recovery. For example, when receiving a pulse that is higher than the threshold of the inverter, the set-reset (SR) latch produces the falling edge of the clock. An on-chip black-out delay generator with a nominal delay of 6.8 ms is inserted to delay the reset of the SR latch, effectively preventing the clock from being retriggered by pulses in the same pulse packet. The SR-latch reset triggers the rising edge of the clock. The inverter, as well as all other gates that directly connect to V_{PZ+}, can be implemented with thick oxide devices and are further protected by on-chip ESD diodes to ensure voltage compliance.

To support the data downlink, it can be important and/or beneficial to measure the duration of one of the pulses in the pulse packet. For example, it is possible to count the number of cycles with the counter 392 in the first pulse that meet the threshold for clock recovery within each pulse packet (shown in FIG. 3f). Decision logic 393 can then converts this count value into a 1 or 0 based on historical count values.

The uplink data appears as a frame-to-frame change in intensity within the reconstructed image, which results from a change in the backscattered echo. To implement this exemplary backscatter ASK, the exemplary circuits can modulate the effective acoustic impedance of the transducer by changing the electrical load attached to it. For example, it is possible to maximize the modulation depth by shorting the piezoelectric element through MBS (see FIG. 3g) when transmitting an uplink bit 0.

This exemplary approach provides three issues which can be addressed in the exemplary design according to the exemplary embodiments of the present disclosure. First, when the transducer is shorted, no clock generation occurs from the pulse packet in the given frame. This missing clock cycle provides no mechanism to synchronously recover from sending a 0. To rectify this issue, the backscatter modulator according to the exemplary embodiment of the present disclosure can asynchronously reset any bit 0 to 1. This exemplary approach extends each bit 0 to take two clock cycles. To maintain a data-independent frame length, each bit 1 is also artificially extended to take two cycles, such that the uplink data rate is data-independent at one half of the frame rate. Second, no downlink data can be recovered when an uplink 0 is transmitted; this precludes full-duplex operation. Third, the power harvesting is uplink-data dependent. As a result, the implant should be able to operate in the worst case at half the maximum power that would otherwise be harvestable for the case of a continuous string of 0's being sent in the uplink channel.

Exemplary Link Layer and Application Layer. To implement the digital control of the chip, a low-leakage standard-cell library according to an exemplary embodiment of the present disclosure can be employed. To facilitate a unique downlink communication to multiple implants within the same FoV, an identification generator can be embedded in the application layer that assigns a unique identification sequence to each mote, implemented with, e.g., an eight-bit fuse array.

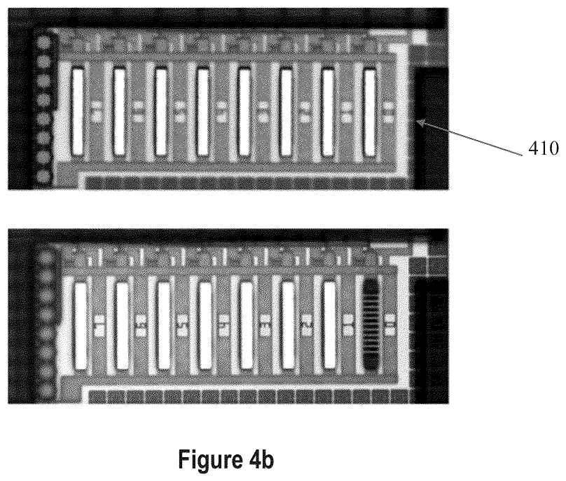

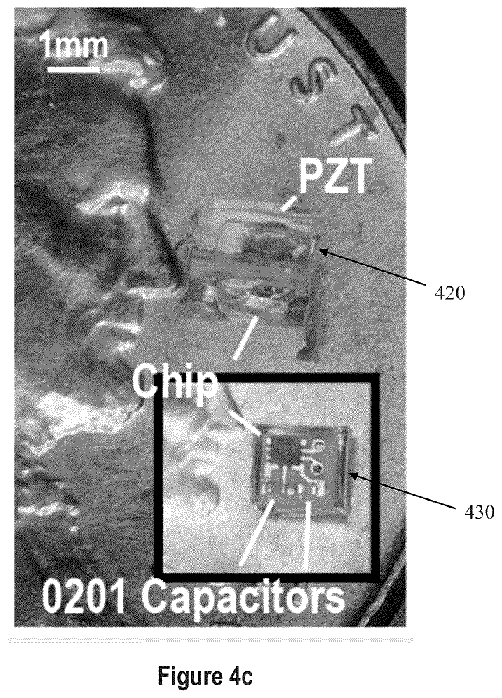

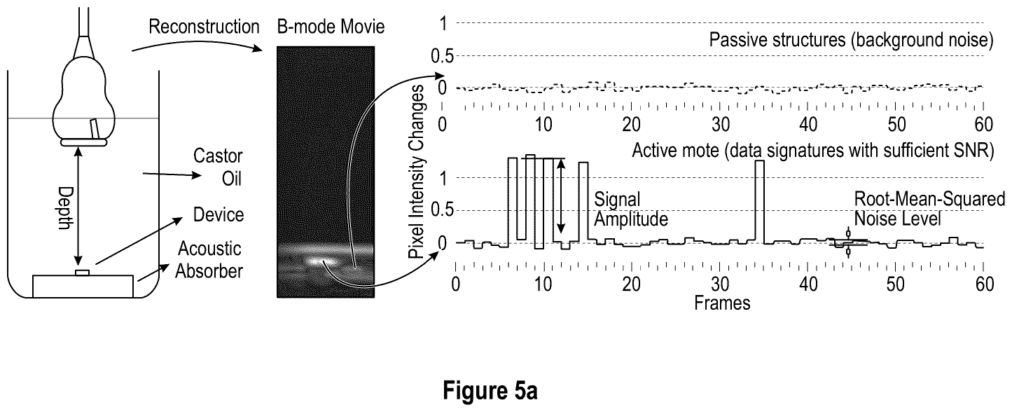

Exemplary Post Processing and Packaging. The exemplary mote ASIC 405 can be fabricated or otherwise provided using, e.g., a TSMC 180 nm MSRFG 1P6M process, occupying a volume of, e.g., 830 μm×740 μm×300 μm (shown in FIG. 4a). Fuses 410 at the bottom right part of the ASIC 405 can be etched to assign an implant identification number different from the default value of 0xFF (see FIG. 4b, an intact fuse indicates a bit 1, otherwise, it indicates bit zero). After this post processing step/procedure, the chips can be packaged into a fully integrated implant by adding two 100-pF decoupling capacitors that measures 0.6 mm×0.3 mm×0.3 mm (e.g., 0201, Murata Manufacturing Co., Ltd) for each of the V_{CC} and V_{DD} power domains (C_{VCC} and C_{VDD}, respectively), as shown in FIG. 3a, and one 1 mm×1 mm×0.5 mm lead zirconate titanate (PZT 5A, Piezo system) piezoelectric crystal 420 (see FIG. 4c) on a 2 mm×2 mm×0.66 mm printed circuit board (PCB) 430. Further size minimizations or reductions are using, e.g., monolithic integration³¹.

Exemplary Performance Characterization. Initial electrical testing can be performed to verify the functionality of the ASIC. To stay fully functioning, the exemplary chip can use preferably, e.g., 57 pW of static power when the dc output of the rectifier reaches 1.2 V. Achieving such ultra-low power consumption can facilitate a higher acoustic energy attenuation, supporting mote operation in deeper tissue.

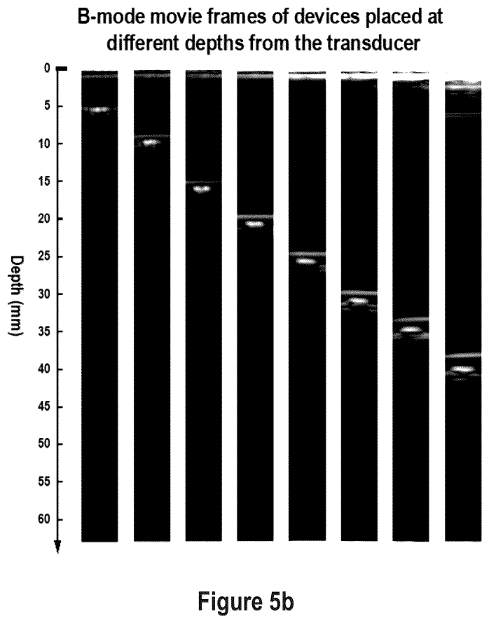

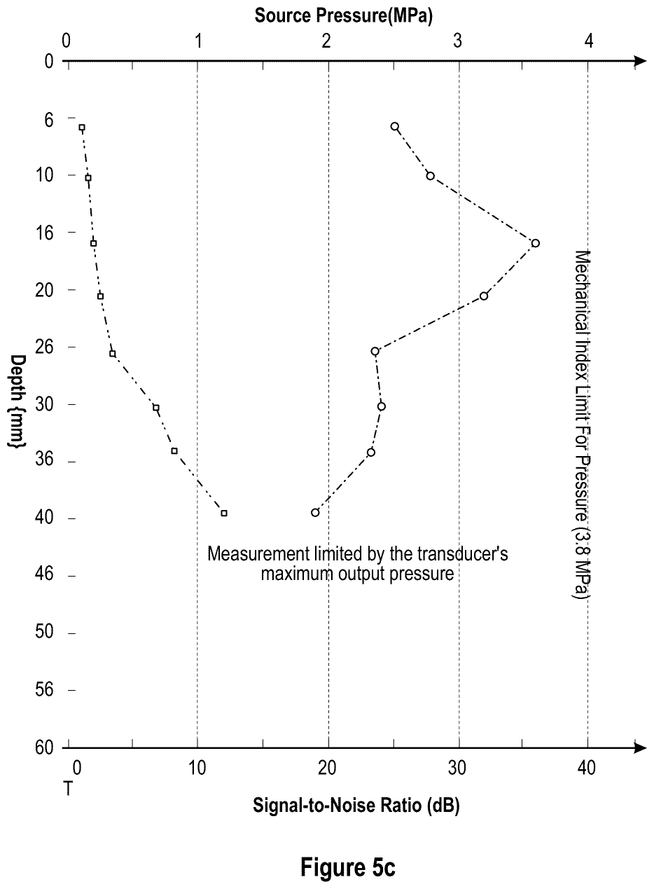

For example, for ultrasonography, a Verasonics Vantage 256 research ultrasound imaging systems can be connected to an L12-3V (Verasonics Inc.), a 192-element linear array probe. FIG. 5a shows an setup according to an exemplary embodiment of the present disclosure, illustrating pixel intensity changes at passive structures and the active mote across frames in the reconstructed B-mode movie. According to such exemplary embodiment of the present disclosure, e.g., 31 elements can be selected and phased to generate focused beams at a 4.0323-MHz center frequency and 1-μs average pulse duration. 192 beams can be scanned on 0.2 mm steps in x direction at a 100-μs interval, generating a 38.6 mm image width. For each beam, backscatter can be recorded for 1280 samples at 15.625 MSamples/s, resulting in an 81.9 μs recording time. For a sound speed of cₛ=1540 m/s, this can give a 63.1 mm maximum image depth. An 800 μs wait time can be inserted between frames to create an imaging session running at 50 fps. For initial characterization, the motes can be immersed in castor oil (Sigma Aldrich) that has an attenuation factor of 7.21 dB/cm at 4 MHz³³, to better emulate acoustic power attenuation in lossy tissue. A custom-implemented delay-and-sum procedure can be used in image reconstruction for uplink data identification.

To harvest sufficient power and deliver detectable uplink modulation depth, the mote can require the focused pulse amplitude in a pulse packet to reach approximately, e.g., 400 kPa, at which point each pulse provides an input energy of approximately, e.g., 52.8 nJ. For example, motes can start-up after power regulation is complete, which can typically utilize about 235 frames or 4.7 seconds. In the exemplary reconstructed floating-point-valued B-mode image 510s, the uplink data sent from the mote can lead to a significant, regular brightness change, which aids in mote localization. The ratio between the signal power and the noise power, or signal-to-noise ratio (SNR), varies as the mote moves away from the source transducer, shown qualitatively in FIGS. 5b and 5c. In particular, FIG. 5b illustrates exemplary B-mode movie frames for depth characterization. FIG. 5c shows exemplary graphs of the exemplary minimum source pressure required to properly power the mote at these depths and the detected signal-to-noise ratio in the corresponding B-mode movies.

With, e.g., a fixed 15-mm focal depth, the mote can stay fully functioning with an uplink SNR of at least 19.1 dB up to 40 mm away from the transducer array. The SNR for the uplink data as rendered in the reconstructed image is higher than that produced by measuring the echo at a single transducer because the delay-and-sum algorithm utilizes the spatial correlation of echo data receive from multiple elements. The 40-mm depth is practically limited by the maximum voltage that can be applied to the piezoelectric elements on the L12-3V probe to generate ultrasound pressure waves. Still, this maximum pressure is much lower than the safety limit for human use as regulated by the “mechanical index” (MI) threshold of 1.9 kPa/\sqrt{Hz}¹⁰, or 3.8 MPa at 4 MHz, beyond which tissue damage can occur. The corresponding ultrasound power is only 9.2 mW/cm², much lower than the 720 mW/cm² safety limit^{10,12}.

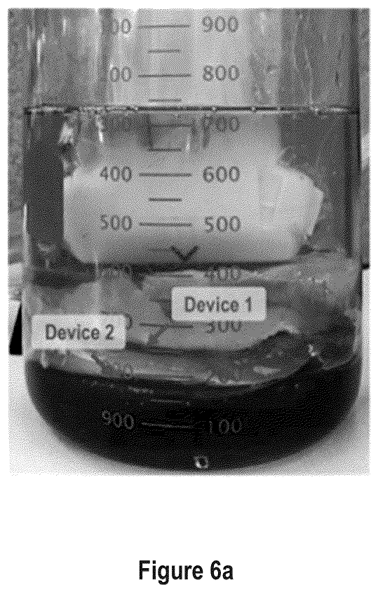

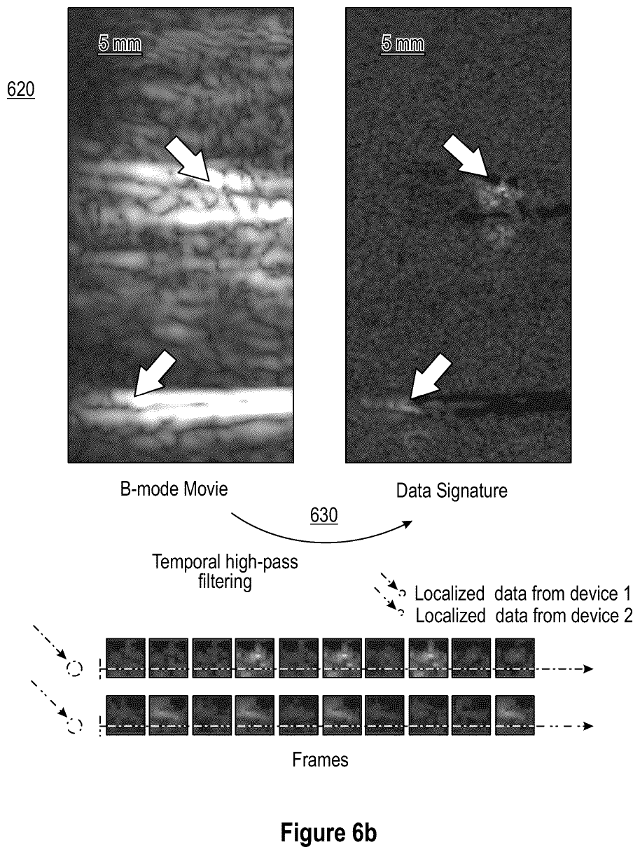

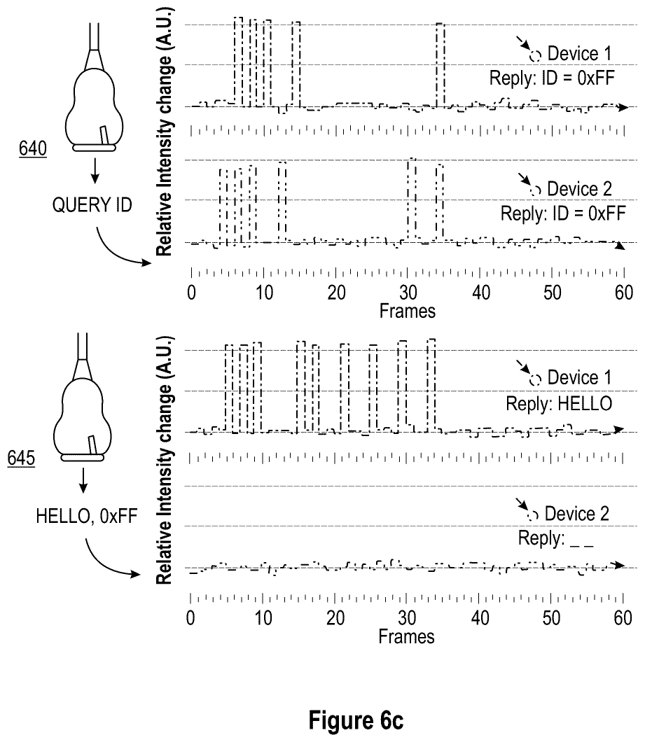

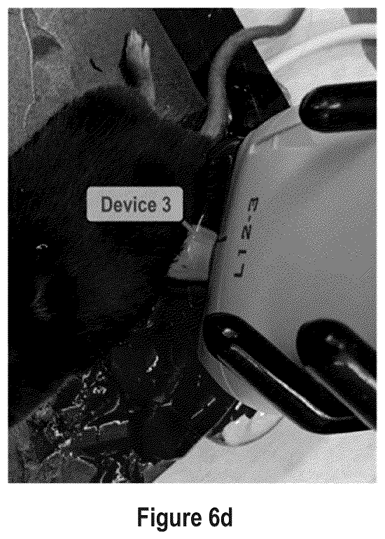

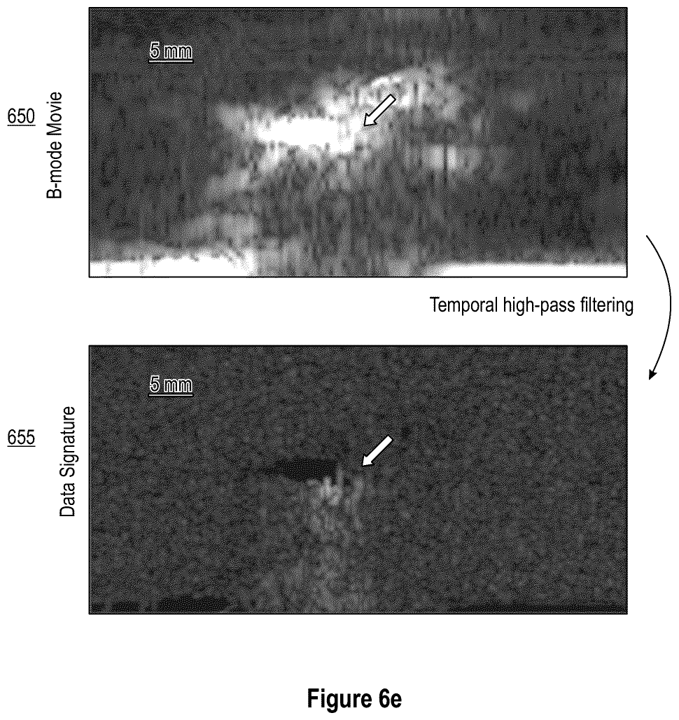

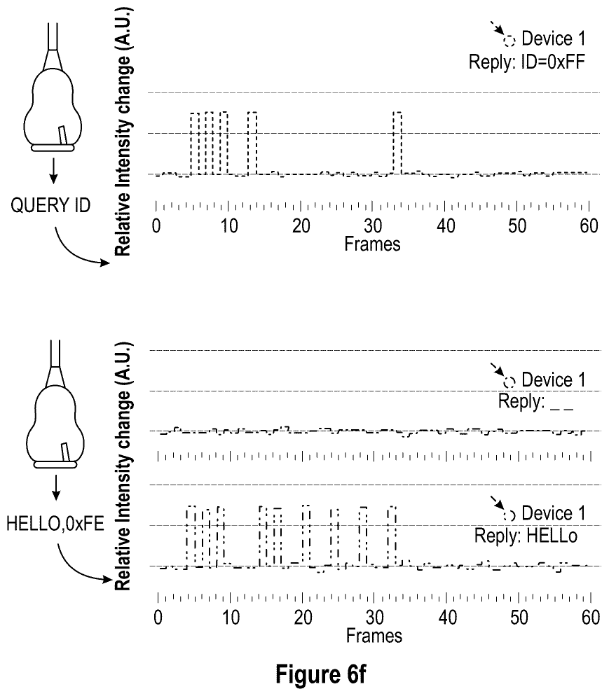

Exemplary Ultrasonography with Phantom. To demonstrate the functioning of the motes within an imaging application, chicken breasts are hand sliced into about 1-cm-thick layers and soaked in castor oil to act as a tissue phantom, with two motes between the chicken-breast layers (see FIG. 6a). For interference-free operations, the motes can be separated by at least the beam width of, e.g., 1 mm and are not stacked vertically to avoid the top mote blocking the backscattered data from the bottom one.

FIG. 6b shows an exemplary B-mode image 620 captured by the linear array transducer according to an exemplary embodiment of the present disclosure. For example, a high-pass temporal filtering 630 of the B-mode movie can be used to amplify the presence of the motes. Once the motes' locations are known, localized intensity changes can be analyzed to extract the uplink data. In this exemplary case, the transducer array can first transmit a “QUERY ID” instruction 640, to which all the motes respond by sending their specific identifiers (IDs) back, as shown in FIG. 6c. Indeed, FIG. 6c shows an exemplary timing diagram regarding instructions sent from the transducer and the motes' replies (in frame-domain, a detected change implies a bit “0”, information translated according to Table S3/S4). This exemplary instruction is designed to be non-device-specific, a fast way to track all the active motes implanted within the FoV, which also enables direct instruction addressing to a specific mote. To verify device specific operations, the simplest identification-sensitive instruction “HELLO” 645 can be transmitted to each of the devices, with the just-captured ID as the argument of the instruction. Interference-free replies are then captured from the received echoes.

For example, in this exemplary case, it can be difficult to visually identify the location of the motes directly from the B-mode movie, as this miniaturized implant is not easily distinguished from other structures in this complicated mechanical environment. However, as active devices, they can present temporal modulation far stronger than the baseline noise level, with a greater than 23.4-dB SNR from the mote placed 15.1 mm away from the transducer, and a greater than 22.1-dB SNR from the mote placed 26.5 mm deep, for all received uplink data. This exemplary property can facilitate an easy location of multiple motes, as well as robust communication with multiple motes simultaneously, as shown in FIG. 6c.

Exemplary In Vivo Demonstration. To demonstrate the functioning of the mote in vivo, the exemplary devices can be implanted into the lower hind limb of a mouse, while the same B-mode imaging system and imaging configuration used for the phantom experiment can be used to communicate with the mote (as shown in FIG. 6d). An acoustic absorber is placed below the hind limb to reduce excess ultrasound reflection from the thermal pad underneath, mimicking imaging in a larger animal in which ultrasound power attenuates beyond the field of view. Modulated backscatter is evident at the location of the mote. By overlaying the extracted data signature 655 within the original B-mode movie 650 (as shown in FIG. 6e), the motes can be tracked in a biogeographic aware fashion; that is, not only it is possible to retrieve the absolute location of such devices, but it is also possible determine its location relative to surrounding structures. FIG. 6f shows an exemplary detailed timing diagram showing the interaction between the imaging system and the implanted mote which is provided to the diagram of FIG. 3c The SNR for this data exceeds 28.5 dB from the mote placed 10.8 mm away from the source linear transducer.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification and drawings, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### EXEMPLARY REFERENCES

The following reference is hereby incorporated by references, in their entireties:
1. Wild, J. J. & Reid, J. M. Application of Echo-Ranging Techniques to the Determination of Structure of Biological Tissues. Science 115, 226-230 (1952).
2. Culjat, M. O., Goldenberg, D., Tewari, P. & Singh, R. S. A Review of Tissue Substitutes for Ultrasound Imaging. Ultrasound Med. Biol. 36, 861-873 (2010).
3. Goss, S. A., Johnston, R. L. & Dunn, F. Comprehensive compilation of empirical ultrasonic properties of mammalian tissues. J. Acoust. Soc. Am. 64, 423-457 (1978).
4. Weber, M. J. et al. A Miniaturized Single-Transducer Implantable Pressure Sensor With Time-Multiplexed Ultrasonic Data and Power Links. IEEE J. Solid-State Circuits 53, 1089-1101 (2018).
5. Piech, D. K. et al. A wireless millimetre-scale implantable neural stimulator with ultrasonically powered bidirectional communication. Nat. Biomed. Eng. 4, 207-222 (2020).
6. Charthad, J. et al. A mm-Sized Wireless Implantable Device for Electrical Stimulation of Peripheral Nerves. IEEE Trans. Biomed. Circuits Syst. 12, 257-270 (2018).
7. Kim, A. et al. An Implantable Ultrasonically-Powered Micro Light Source (μLight) for Photodynamic Therapy. Sci. Rep. 9, (2019).
8. Sonmezoglu, S., Fineman, J. R., Maltepe, E. & Maharbiz, M. M. Monitoring deep-tissue oxygenation with a millimeter-scale ultrasonic implant. Nat. Biotechnol. 39, 855-864 (2021).
9. Thimot, J. & Shepard, K. L. Bioelectronic devices: Wirelessly powered implants. Nat. Biomed. Eng. 1, 0051 (2017).
10. Marketing Clearance of Diagnostic Ultrasound Systems and Transducers. 64 https://www.fda.gov/media/71100/download (2017).
11. Agrawal, D. R. et al. Conformal phased surfaces for wireless powering of bioelectronic microdevices. Nat. Biomed. Eng. 1, 0043 (2017).
12. Chang, T. C., Wang, M. L., Charthad, J., Weber, M. J. & Arbabian, A. 27.7 A 30.5 mm³ fully packaged implantable device with duplex ultrasonic data and power links achieving 95 kb/s with <10⁻⁴ BER at 8.5 cm depth. in 2017 IEEE International Solid-State Circuits Conference (ISSCC) 460-461 (IEEE, 2017). doi:10.1109/ISSCC.2017.7870460.
13. Al-Kalbani, A. I., Yuce, M. R. & Redoute, J.-M. Design Methodology for Maximum Power Transmission, Optimal BER-SNR and Data Rate in Biomedical Implants. IEEE Commun. Lett. 17, 1897-1900 (2013).
14. Shi, C., Costa, T., Elloian, J., Zhang, Y. & Shepard, K. L. A 0.065-mm³ Monolithically-Integrated Ultrasonic Wireless Sensing Mote for Real-Time Physiological Temperature Monitoring. IEEE Trans. Biomed. Circuits Syst. 14, 412-424 (2020).
15. Ghanbari, M. M. et al. A Sub-mm³ Ultrasonic Free-Floating Implant for Multi-Mote Neural Recording. IEEE J. Solid-State Circuits 54, 3017-3030 (2019).
16. Meng, M. & Kiani, M. Self-Image-Guided Ultrasonic Wireless Power Transmission to Millimeter-Sized Biomedical Implants. in 2019 41st Annual International Conference of the IEEE Engineering in Medicine and Biology Society (EMBC) 364-367 (IEEE, 2019). doi: 10.1109/EMBC.2019.8857559.
17. Kashani, Z. & Kiani, M. Optimal Ultrasonic Pulse Transmission for Miniaturized Biomedical Implants. in 2019 IEEE Biomedical Circuits and Systems Conference (BioCAS) 1-4 (IEEE, 2019). doi: 10.1109/BIOCAS.2019.8918704.
18. Benedict, B. C., Ghanbari, M. M., Faraji Alamouti, S., Ersumo, N. T. & Muller, R. Time reversal beamforming for powering ultrasonic implants. in 2021 10th International IEEE/EMBS Conference on Neural Engineering (NER) 647-650 (IEEE, 2021). doi: 10.1109/NER49283.2021.9441162.
2019. Wang, M. L., Chang, T. C. & Arbabian, A. Ultrasonic Implant Localization for Wireless Power Transfer: Active Uplink and Harmonic Backscatter. in 2019 IEEE International Ultrasonics Symposium (IUS) 818-821 (IEEE, 2019). doi: 10.1109/ULTSYM.2019.8926006.
20. Tranquart, F., Grenier, N., Eder, V. & Pourcelot, L. Clinical use of ultrasound tissue harmonic imaging. Ultrasound Med. Biol. 25, 889-894 (1999).
21. Zhang, Y. & Shepard, K. L. A 0.6-mm² Powering and Data Telemetry System Compatible with Ultrasound B-Mode Imaging for Freely Moving Biomedical Sensor Systems. in 2019 IEEE Custom Integrated Circuits Conference (CICC) 1-4 (IEEE, 2019). doi: 10.1109/CICC.2019.8780205.
22. Zhang, Y., Cardoso, F. A. & Shepard, K. L. A 0.72 nW, 1 Sample/s Fully Integrated pH Sensor with 65.8 LSB/pH Sensitivity. in 2020 IEEE Symposium on VLSI Circuits 1-2 (IEEE, 2020). doi: 10.1109/VLSICircuits18222.2020.9163023.
23. Truesdell, D. S. & Calhoun, B. H. A 640 pW 22 pJ/sample Gate Leakage-Based Digital CMOS Temperature Sensor with 0.25° C. Resolution. in 2019 IEEE Custom Integrated Circuits Conference (CICC) 1-4 (IEEE, 2019). doi: 10.1109/CICC.2019.8780382.
24. Wang, H. & Mercier, P. P. A 113 pW fully integrated CMOS temperature sensor operating at 0.5 V. in 2017 IEEE SENSORS 1-3 (IEEE, 2017). doi: 10.1109/ICSENS.2017.8234279.
25. Leloup, T., El Kazzi, W., Schuind, F. & Warzee, N. A Novel Technique for Distal Locking of Intramedullary Nail Based on Two Non-constrained Fluoroscopic Images and Navigation. IEEE Trans. Med. Imaging 27, 1202-1212 (2008).
26. Sharma, S. et al. 20.4 3D Surgical Alignment with 100 μm Resolution Using Magnetic-Field Gradient-Based Localization, in 2020 IEEE International Solid-State Circuits Conference—(ISSCC) 318-320 (IEEE, 2020). doi: 10.1109/ISSCC19947.2020.9063108.
27. Diagnostic ultrasound: physics and equipment. (CRC Press/Taylor & Francis Group, 2019).
28. Charthad, J., Weber, M. J., Chang, T. C. & Arbabian, A. A mm-Sized Implantable Medical Device (IMD) With Ultrasonic Power Transfer and a Hybrid Bi-Directional Data Link. IEEE J. Solid-State Circuits 50, 1741-1753 (2015).
29. Chang, T. C. et al. Design of Tunable Ultrasonic Receivers for Efficient Powering of Implantable Medical Devices With Reconfigurable Power Loads. IEEE Trans. Ultrason. Ferroelectr. Freq. Control 63, 1554-1562 (2016).
30. Ramadass, Y. K. & Chandrakasan, A. P. An Efficient Piezoelectric Energy Harvesting Interface Circuit Using a Bias-Flip Rectifier and Shared Inductor. IEEE J. Solid-State Circuits 45, 189-204 (2010).
31. Shi, C. et al. Application of a sub-0.1-mm3 implantable mote for in vivo real-time wireless temperature sensing. Sci. Adv. (2021)
32. Qing Dong, Kaiyuan Yang, Blaauw, D. & Sylvester, D. A 114-pW PMOS-only, trim-free voltage reference with 0.26% within-wafer inaccuracy for nW systems. in 2016 IEEE Symposium on VLSI Circuits (VLSI-Circuits) 1-2 (IEEE, 2016). doi: 10.1109/VLSIC.2016.7573494.
33. Ping He. Experimental verification of models for determining dispersion from attenuation. IEEE Trans. Ultrason. Ferroelectr. Freq. Control 46, 706-714 (1999).
34. Aklimi, E., Piedra, D., Tien, K., Palacios, T. & Shepard, K. L. Hybrid CMOS/GaN 40-MHz Maximum 20-V Input DC-DC Multiphase Buck Converter. IEEE J. Solid-State Circuits 52, 1618-1627 (2017).
35. Madore, B. & Meral, F. C. Reconstruction algorithm for improved ultrasound image quality. IEEE Trans. Ultrason. Ferroelectr. Freq. Control 59, 217-230 (2012). 5
36. Peterson, D. K. & Kino, G. S. Real-Time Digital Image Reconstruction: A Description of Imaging Hardware and an Analysis of Quantization Errors. IEEE Trans. Sonics Ultrason. 31, 337-351 (1984).

1. An apparatus for use with ultrasound imaging, comprising:
at least one implantable device which is configured to:
communicate with an ultrasound imaging system,
be located by the ultrasound imaging system using ultrasound signals generated thereby to generate a location of the at least one implantable device, and
transmit data to the ultrasound imaging system from the location of the at least one implantable device that was located by the ultrasound imaging system.

2. The apparatus of claim 1, wherein the at least one implantable device is further configured to receive further data from the ultrasound imaging system.

3. The apparatus of claim 1, wherein the at least one implantable device is further configured to be powered by the ultrasound imaging system.

4. The apparatus of claim 3, wherein the at least one implantable device is further configured to be continuously powered by the ultrasound imaging system when the at least one implantable device is located within a field of view of the ultrasound imaging system.

5. The apparatus of claim 1, wherein the at least one implantable device transmits the data to the ultrasound imaging system using modulation of the ultrasound wave.

6. The apparatus of claim 1, wherein the at least one implantable device is further configured to be implanted into a body through an injection.

7. The apparatus of claim 6, wherein the at least one implantable device is sized and configured to be injected into the body using a syringe.

8. The apparatus of claim 1, wherein the at least one implantable device is further configured to receive ultrasound signals from the ultrasound imaging system so that the ultrasound imaging system ascertains the location of the at least one implantable device.

9. The apparatus of claim 8, wherein the transmission of the data to the ultrasound imaging system and the receipt of the ultrasound signals are synchronized.

10. The apparatus of claim 8, wherein the receipt of the ultrasound signals by the at least one implantable device from the ultrasound imaging system is performed through modulation of the ultrasound wave.

11. The apparatus of claim 10, wherein the further modulation of the received ultrasound signals is performed using at least one amplitude shift key, and wherein the modulation of the transmission of the data is performed using at least one load shift key back.

12. The apparatus of claim 1, wherein the at least one implantable device is a CMOS device.

13. The apparatus of claim 1, wherein the at least one implantable device has a monolithically-integrated piezoelectric transducer.

14. The apparatus of claim 1, wherein the at least one implantable device is a plurality of implantable devices.

15. The apparatus of claim 14, wherein each of the plurality of the implantable devices are deployable within a field of view of the ultrasound imaging system and providing a non-interfering parallel operation.

16. The apparatus of claim 14, wherein each of the plurality of the implantable devices are configured to be implanted at different locations within a body.

17. The apparatus of claim 1, wherein the transmission of the data from the at least one implantable device to the ultrasound imaging system is performed using a backscattered acoustic energy.

18. The apparatus of claim 1, wherein the data transmitted to the ultrasound imaging system includes information regarding at least one area of a body on which the at least one implantable devices is provided.

19. A method for performing ultrasound imaging, comprising:
implanting, into a structure, at least one device which is configured to be responsive to ultrasound signals generated by the ultrasound imaging system;
locating the at least one device within the structure by the ultrasound imaging system using the ultrasound signals to generate a location of the at least one device within the structure; and
transmitting data to the ultrasound imaging system from the location of the at least one implantable device that was located by the ultrasound imaging system.

20. The method of claim 19, further comprising powering the at least one device by the ultrasound imaging system.

21. The method of claim 20, wherein the at least one device is continuously powered by the ultrasound imaging system when the at least one device is located within a field of view of the ultrasound imaging system.

22. The method of claim 19, further comprising transmitting the data to the ultrasound imaging system by the at least one device using modulation.

23. The method of claim 19, wherein the at least one implantable device is implanted into the structure through an injection using a syringe.

24-35. (canceled)
